(19) United States
(12) Patent Application Publication
(10) Pub. No.: US 2005/0228404 A1
(43) Pub. Date: Oct. 13, 2005

(54) Surgical navigation system component automated imaging navigation and related processes

(76) Inventor: Dirk Vandevelde, Kontich (BE)

Correspondence Address: CHIEF PATENT COUNSEL;SMITH & NEPHEW, INC., 1450 BROOKS ROAD, MEMPHIS, TN, 38116, US

(21) Appl. No.: 10/823,343

(22) Filed: Apr. 12, 2004

(51) Int. Cl. A61B 19/00

(52) U.S. Cl. 606/130

(57) ABSTRACT

Systems and processes for use in computer aided or computer navigated surgery include probes with indicia which define axes relative to which images are desired. Computer functionality generates and stores the position and location of these indicia. After the indicia have been registered into the system, imaging apparatus may be moved, manually or automatically, into the correct position to capture the desired image. In various embodiments, probes may be left in place during, or removed prior, to imaging. In addition, several axes may be defined, and their location and position data generated and stored, so that the imaging device may move into each position in turn to capture a series of desired images.

### FIELD OF THE INVENTION

The present invention relates to imaging alignment systems for use in surgical navigation, and methods for their use. More specifically, the invention relates to a system for navigating the position of imaging equipment to a specific location previously defined by a user in order to provide images of specific anatomy in specific locations and/or orientations.

### BACKGROUND

A major concern during surgical procedures as well as other medical operations is carrying out the procedures with as much precision as possible. For example, in orthopedic procedures, less than optimum alignment of implanted prosthetic components may cause undesired wear and revision, which may eventually lead to the failure of the implanted prosthesis. Other general surgical procedures also require precision in their execution.

With orthopedic procedures, for example, previous practices have not allowed for precise alignment of prosthetic components. For example, in a total knee arthroplasty, previous instrument design for resection of bone limited the alignment of the femoral and tibial resections to average value for varus/valgus, flexion/extension and external/internal rotation. Additionally, surgeons often use visual landmarks or “rules of thumb” for alignment which can be misleading due to anatomical variability. Intramedullary referencing instruments also violate the femoral and tibial canal. This intrusion increases the risk of fat embolism and unnecessary blood loss in the patient.

Processes according to various embodiments of the present invention are applicable not only for knee repair, reconstruction or replacement surgery, but also repair, reconstruction or replacement surgery in connection with any other joint of the body as well as any other surgical or other operation where it is useful to track position and orientation of body parts, non-body components and/or virtual references such as rotational axes, and to display and output data regarding positioning and orientation of them relative to each other for use in navigation and performance of the operation.

Several manufacturers currently produce image-guided surgical navigation systems that are used to assist in performing surgical procedures with greater precision. The TREON™ and iON™ systems with FLUORONAV™ software manufactured by Medtronic Surgical Navigation Technologies, Inc. are examples of such systems. The BrainLAB VECTORVISION™ system is another example of such a surgical navigation system. Systems and methods for accomplishing image-guided surgery are also disclosed in U.S. Ser. No. 10/364,859, filed Feb. 11, 2003 and entitled “Image Guided Fracture Reduction,” which claims priority to U.S. Ser. No. 60/355,886, filed Feb. 11, 2002 and entitled “Image Guided Fracture Reduction”; U.S. Ser. No. 60/271,818, filed Feb. 27, 2001 and entitled “Image Guided System for Arthroplasty”; U.S. Ser. No. 10/229,372, filed Aug. 27, 2002 and entitled “Image Computer Assisted Knee Arthroplasty”; U.S. Ser. No. 10/084,278 filed Feb. 27, 2002 and entitled “Total Knee Arthroplasty Systems and Processes,” which claims priority to provisional application entitled “Surgical Navigation Systems and Processes,” Ser. No. 60/355,899, filed Feb. 11, 2002; U.S. Ser. No. 10/084,278 filed Feb. 27, 2002 and entitled “Surgical Navigation Systems and Processes for Unicompartmental Knee Arthroplasty,” which claims priority to provisional application entitled “Surgical Navigation Systems and Processes,” Ser. No. 60/355,899, filed Feb. 11, 2002; U.S. Ser. No. 10/084291 entitled Surgical Navigation Systems and Processes for High Tibial Osteotomy,” which claims priority to provisional application entitled “Surgical Navigation Systems and Processes,” Ser. No. 60/355,899, filed Feb. 11, 2002; provisional application entitled “Image-guided Navigated Precisions Reamers,” Ser. No. 60/474,178, filed May 29, 2003; nonprovisional application entitled “Surgical Positioners,” T. Russell, P. Culley, T. Ruffice, K. Raburn and L. Grisoni, inventors, filed Oct. 3, 2003; and nonprovisional application entitled Surgical Navigation System Component Fault Interfaces and Related Processes, R. Thornberry and J. Stallings, inventors, filed Oct. 20, 2003; the entire contents of each of which are incorporated herein by reference as are all documents incorporated by reference therein.

These systems and processes use position and/or orientation tracking sensors such as infrared sensors acting stereoscopically or other sensors acting in conjunction with reference structures or reference transmitters to track positions of body parts, surgery-related items such as implements, instrumentation, trial prosthetics, prosthetic components, and virtual constructs or references such as rotational axes which have been calculated and stored based on designation of bone landmarks. Processing capability such as any desired form of computer functionality, whether standalone, networked, or otherwise, takes into account the position and orientation information as to various items in the position sensing field (which may correspond generally or specifically to all or portions or more than all of the surgical field) based on sensed position and orientation of their associated reference structures such as fiducials, reference transmitters, or based on stored position and/or orientation information. The processing functionality correlates this position and orientation information for each object with stored information, such as a computerized fluoroscopic imaged file, a wire frame data file for rendering a representation of an instrument component, trial prosthesis or actual prosthesis, or a computer generated file relating to a rotational axis or other virtual construct or reference. The processing functionality then displays position and orientation of these objects on a screen or monitor, or otherwise. Thus, systems or processes, by sensing the position of reference structures or transmitters, can display or otherwise output useful data relating to predicted or actual position and orientation of body parts, surgically related items, implants, and virtual constructs for use in navigation, assessment, and otherwise performing surgery or other operations.

Some of these reference structures or reference transmitters may emit or reflect infrared light that is then detected by an infrared camera. The references may be sensed actively or passively by infrared, visual, sound, magnetic, electromagnetic, x-ray or any other desired technique. An active reference emits energy, and a passive reference merely reflects energy. Reference structures may have at least three, but usually four, markers or fiducials that are traced by an infrared sensor to determine the position and orientation of the reference and thus the position and orientation of the associated instrument, implant component or other object to which the reference is attached.

In addition to reference structures with fixed fiducials, modular fiducials, which may be positioned independent of each other, may be used to reference points in the coordinate system. Modular fiducials may include reflective elements which may be tracked by two, sometimes more sensors whose output may be processed in concert by associated processing functionality to geometrically calculate the position and orientation of the item to which the modular fiducial is attached. Like fixed fiducial reference structures, modular fiducials and the sensors need not be confined to the infrared spectrum—any electromagnetic, electrostatic, light, sound, radio frequently or other desired technique may be used. Similarly, modular fiducials may “actively” transmit reference information to a tracking system, as opposed to “passively” reflecting infrared or other forms of energy.

Some image-guided surgical navigation systems allow reference structures to be detected at the same time the fluoroscopy imaging is occurring. This allows the position and orientation of the reference structure to be coordinated with the fluoroscope imaging. Then, after processing position and orientation data, the reference structures may be used to track the position and orientation of anatomical features that were recorded fluoroscopically. Computer-generated images of instruments, components, or other structures that are fitted with reference structures may be superimposed on the fluoroscopic images. The instruments, trial, implant or other structure or geometry can be displayed as 3-D models, outline models, or bone-implant interface surfaces.

Some image-guided surgical navigation systems monitor the location and orientation of the reference structures and consequently the portion of the anatomy or instruments secured to the reference structure by either actively or passively detecting the position of fiducials associated with the reference structure. Because the fiducials may be arranged in particular patterns, the system can determine the exact orientation and location of the reference structure associated with the fiducials. In other words, depending upon the particular location of the individual fiducials, the system will “see” the reference structure in a particular way and will be able to calculate the location and orientation of the reference structure based upon that data. Consequently, the system can determine the exact orientation and location of the portion of the anatomy or instrument associated with the reference structure.

Once a reference structure has been located by an image-guided system, and placed on its coordinate system, the exact location and orientation of the reference structure can be stored in the navigation system. Thus, it may be physically removed from or relocated within the system while its original position and orientation are retained.

When acquiring fluoroscopic images for navigated surgery, it frequently requires multiple images to center on the specific anatomy that needs to be imaged. While the correct orientation and position of a desired image may be known to a surgeon, it can take several iterative manipulations of an imaging device, and several images, in order to successfully capture the desired fluoroscopic image. This lengthens the time necessary to complete the surgical procedure and can result in unnecessary complications resulting from the additional length of time the patient is in surgery. In addition, this results in increased radiation exposure which can lead to obvious dangers.

### SUMMARY

Various aspects and embodiments of the present invention include processes by which a surgeon, or other surgery attendant, may obtain a desired image by indicating a desired axis of view using an image guided probe.

According to one aspect of the present invention, a user captures a desired image by registering a patient within a coordinate system and indicating a desired axis with an image guided probe. An image is then taken along the desired axis by an imaging apparatus.

According to another aspect of the present invention, a user captures a desired image by registering a patient within a coordinate system and indicating a desired axis with an image guided probe. The navigation system stores the position and location for the desired image axis within the computer functionality. The imaging apparatus, using the stored axis information, moves to the correct position and the desired image is taken.

According to another aspect of the present invention, a user indicates several axes on which he would like images taken by indicating several desired axes with image guided probes. The imaging apparatus then takes the images along the desired axes.

According to other aspects of the present invention, a user indicates several axes on which he would like images taken, indicating the desired axis with an image guided probe, prompting the computer to store the axis information within its functionality, relocating the image guided probe to another axis along which he would like an image taken and prompting the computer to store this information. This process continues until the user has indicated all of the axes along which he would like images taken. The imaging apparatus, using the stored axes data, moves sequentially into the correct positions taking images along the desired axes.

### BRIEF DESCRIPTION

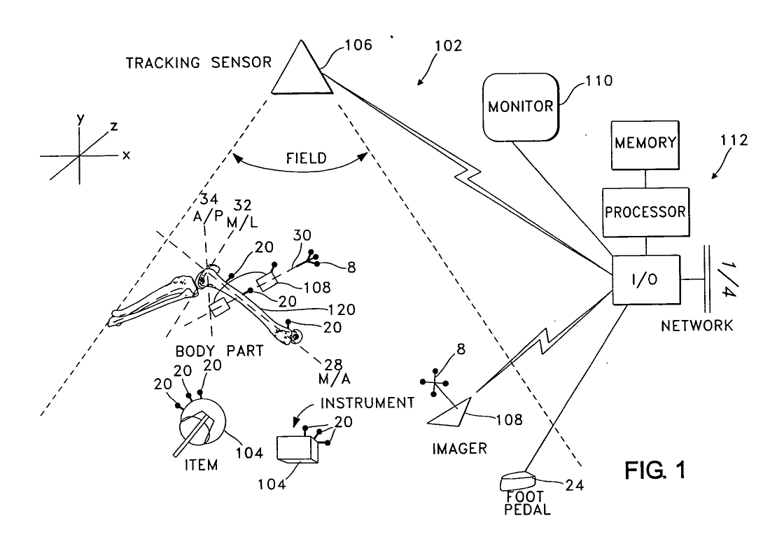

FIG. 1 shows a schematic view of a tracking system according to one embodiment of the present invention.

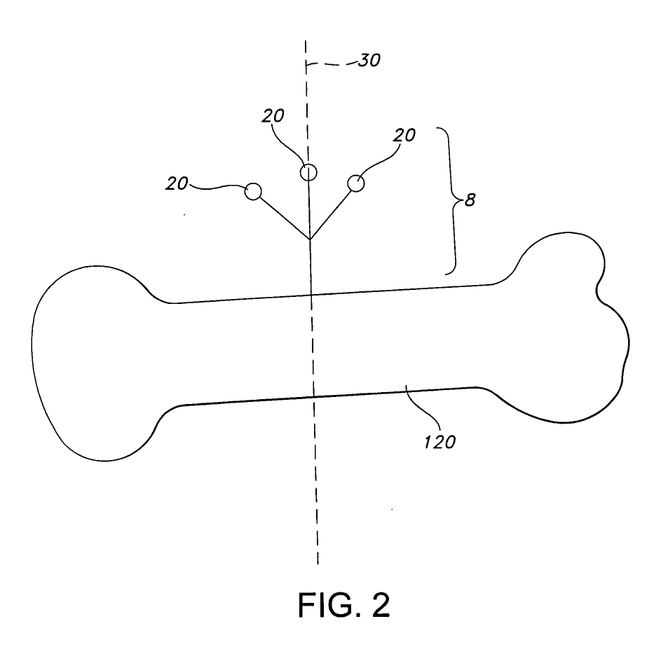

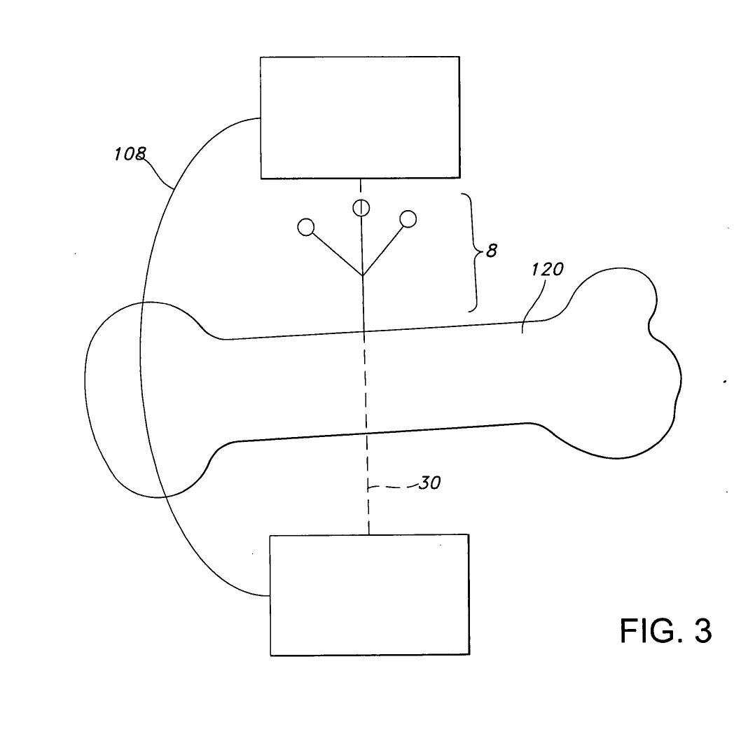

FIG. 2 shows a schematic view of a probe placed on a body part along a desired axis according to one embodiment of the present invention.

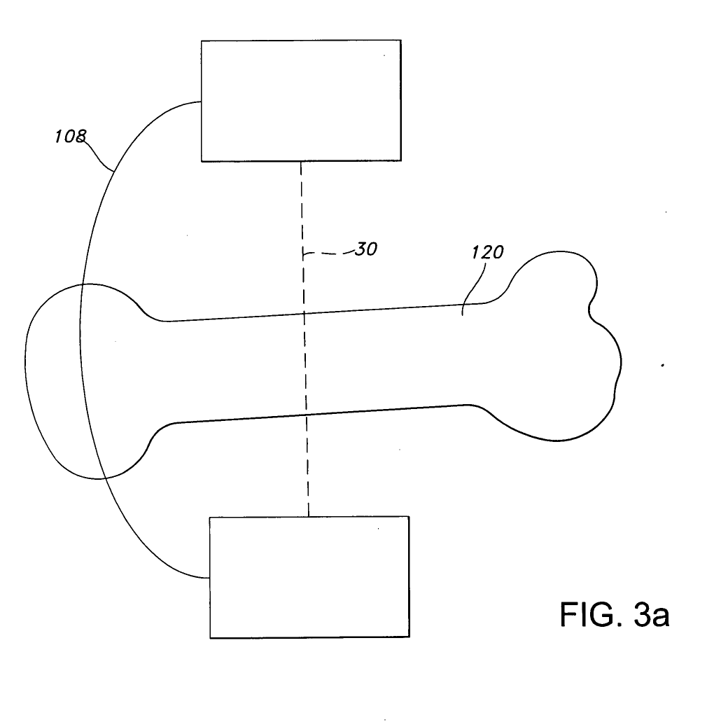

FIG. 3 shows a schematic view of an imaging apparatus positioned to image the desired axis of FIG. 2.

FIG. 3a shows a schematic view of the imaging apparatus positioned to image the desired axis of FIG. 2 after the probe has been removed.

### DETAILED DESCRIPTION

FIG. 1 is a schematic view showing one embodiment of a system according to the present invention. In the embodiment shown in FIG. 1, indicia 20 are structural frames, some of which contain reflective elements, some of which contain LED active elements, some of which can contain both, for tracking using stereoscopic infrared sensors suitable, at least operating in concert, for sensing, storing, processing and/or outputting data relating to (“tracking”) position and orientation of indicia 20 and thus items 104 or body parts 120 to which they are attached or otherwise associated. Position sensor 106 may be any sort of sensor functionality for sensing position and orientation of indicia 20 and therefore items with which they are associated, according to whatever desired electrical, magnetic, electromagnetic, sound, physical, radio frequency, or other active or passive technique.

In the embodiment shown in FIG. 1, computing functionality 112 can include processing functionality, memory functionality, input/output functionality whether on a standalone or distributed bases, via any desired standard, architecture, interface and/or network topology. In this embodiment, computing functionality 112 is connected to a monitor 110 on which graphics and data may be presented to the surgeon during surgery. The screen preferably has a tactile interface so that the surgeon may point and click on screen for tactile screen input in addition to or instead of, if desired, keyboard and mouse conventional interfaces. Additionally, a foot pedal 24 or other convenient interface may be coupled to functionality 112 as can any other wireless or wireline interface to allow the surgeon, nurse, or other desired use to control or direct functionality 112 in order to, among other things, capture position/orientation information when certain components are oriented or aligned properly.

Computer functionality 112 can process, store and output on monitor 110 and otherwise various forms of data which correspond in whole or part to items 104. The computer functionality 112 can also store data relating to configuration, size and other properties of items 104 such as implements, instrumentation, trial components, implant components and other items used in surgery. Additionally, computer functionality 112 can track any point in the position/orientation sensor 106 field such as by using a probe 8. The probe can also contain or be attached to indicia 20. The surgeon, nurse, or other user touches the tip of probe 8 to a point such as a landmark on bone structure and actuates the foot pedal 24 or otherwise instructs the computer 112 to note the landmark position. The position/orientation sensor 106 “sees” the position and orientation of the indicia 20 “knows” where the tip of probe 8 is relative to the indicia 20 and thus calculates and stores, and can display on monitor 110 whenever desired in whatever form or fashion or color, the point or other position designated by probe 8 when the foot pedal 24 is hit or other command is given. Thus, probe 8 can be used to designate landmarks on bone structure in order to allow the computer 112 to store and track, relative to movement of the bone indicia 20, virtual or logical information such as mechanical axis 28, medial lateral axis 32 and anterior/posterior axis 34 of body part 120 in addition to any other virtual or actual construct or reference.

In the embodiment shown in FIG. 1, images of body part 120 are obtained using imaging functionality 108 attached to indicia 20. The probe 8 also has indicia 20 attached. A surgeon aligns the probe 8 along the position of the desired axis 30 for imaging and the foot pedal 24 is activated. The position/orientation sensor 106 “sees” position and orientation of the indicia 20 attached to the body part 120 and also the position and orientation of the indicia 20 attached to the probe 8 whose tip is touching a landmark on body part 104 and thus can calculate the desired axis 30 for imaging. The computer stores the desired axis 30 with this position/orientation information. The imaging functionality 108 with indicia 20 attached then moves to the position and location stored in the computer functionality 112 that was previously defined by the probe 8. An image is then taken along the desired axis 30.

Similarly, the mechanical axis and other axes or constructs of body parts 104 can also be “registered” for tracking by the system and subsequent imaging. The surgeon uses the probe to select any desired anatomical landmarks or references at the operative site. These points are registered in three dimensional space by the system and are tracked relative to the indicia on the patient anatomy. After the mechanical axis and other rotation axes and constructs relating to the body parts are established, imaging apparatus can be used to capture images along these axes.

Additionally, probe 8 can be used to define a plurality of desired axes. A surgeon positions the probe 8 along the desired axis, or to designate the landmark or landmarks along which he would like images taken in sequence. At the site of each desired image, the surgeon activates the foot pedal or other actuator and stores the position and orientation data for each axis in the computer. The computer then uses this stored information to direct the imaging apparatus to the correct location to capture each desired image.

FIGS. 2 and 3 schematically show one embodiment of the present invention. FIG. 2 shows a probe 8 that includes indicia 20 in the form of fiducials. The probe 8 is attached to a body part 120 along an axis 30 for which an image is desired. The probe 8 is positioned to indicate the desired axis 30 along which the image will be taken. FIG. 3 shows the imaging device 108 positioned to capture the desired image of the body part 120 of FIG. 2 along the axis 30 defined by the probe 8. Alternatively, as shown in FIG. 3A, the probe 8 may be removed. The desired axis 30 on which the image is to be taken has been stored in the computer functionality. An imaging apparatus 108, in this embodiment shown as a C-arm, is positioned using the data stored in the computer functionality in the correct position and orientation to capture the image desired by the axis 30 provided by the probe. This positioning can be accomplished manually using information stored in the system, and/or the computer can automatically position the C-arm using information stored in the system, at least some of which includes information generated with the use of probe 8.

While FIGS. 2 and 3 depict one embodiment of the present invention, the invention includes any navigation alignment system which allows a user to establish or input desired axes for images into a computer-aided navigation system through the use of probes which have fiducials sensed by the system.

The foregoing is provided for purposes of disclosure of various aspects and embodiments of the present invention. Changes, deletions, additions or and substitutions may be made to components, combinations, processes, and embodiments disclosed in this document without departing from the scope or spirit of the invention.

1. A computer aided surgery navigation system comprising:
a. a sensor adapted to sense position of a plurality of indicia attached to an item used in surgery;
b. computer functionality adapted to receive information from the sensor about position of the indicia and generate information corresponding to position and orientation of a probe to which the indicia are attached;
c. a probe adapted to be positioned near a body part, said probe attached to at least one indicium, whereby the position and orientation of the probe is capable of being tracked by said computer functionality;
d. imaging functionality attached to at least one indicium, adapted to capture an image of the body part;
e. wherein a desired axis for the image is defined by the probe; and
f. wherein the imaging functionality is adapted to be moved to the correct position and orientation to capture the desired image by alignment with the axis defined by the probe.

2. A system according to claim 1 wherein at least some of the indicia are fiducials.

3. A system according to claim 2 wherein at least some of the fiducials feature reflective surfaces adapted to be sensed by an infrared sensor device.

4. A system according to claim 1 wherein at least some of the indicia are active devices.

5. A system according to claim 4 wherein at least some of the active devices are transponders which emit energy when interrogated.

6. A system according to claim 1 wherein the imaging functionality is manually positioned.

7. A system according to claim 1 wherein the imaging functionality is automatically positioned.

8. A system according to claim 7 wherein the imaging functionality is correctly positioned and oriented using information stored in the computer functionality.

9. A system according to claim 1 wherein the probe has a pointed tip.

10. A system according to claim 9 wherein the desired axis comprises a straight line extending from the tip of the probe.

11. A system according to claim 10 wherein the desired axis comprises:
a) a first point, the position of which is identified to the computer functionality using the probe;
b) at least one more point, the position of which is identified to the computer functionality using the probe; and
c) a line extending through the first and at least one more point generated by the computer functionality.

12. A system according to claim 10, wherein the desired axis is defined by:
a) placing the tip of the probe at a first point along the desired axis;
b) storing the position and orientation information of the first point in the computer functionality;
c) placing the tip of the probe at a second point along the desired axis;
d) storing the position and orientation information of the second point in the computer functionality; and
e) prompting the computer functionality to connect the points.

13. A system according to claim 1 wherein a plurality of probes are positioned near the item, defining a plurality of axes for images.

14. A system according to claim 1 wherein the computer functionality retains the information generated corresponding to the location and position of the probe even after the probe is removed.

15. A system according to claim 14 wherein the imaging functionality captures the desired image after the probe has been removed.

16. A system according to claim 14 wherein the imaging functionality captures a plurality of desired images after the probes have been removed.

17. A system according to claim 1 wherein the imaging functionality is a C-arm fluoroscope.

18. A system according to claim 1 wherein the computer functionality is instructed to capture the position and location of a desired axis through the use of a foot pedal.

19. A computer aided surgery navigation system comprising:
a. an infrared sensor adapted to sense position of a plurality of fiducials attached to an item used in surgery;
b. computer functionality adapted to receive information from the sensor about positions of the indicia and generate information corresponding to position and orientation of the item to which the indicia are attached;
c. a probe adapted to be positioned near a body part, said probe attached to at least one indicium, whereby the position and orientation of the probe is capable of being tracked by said computer functionality;
d. imaging functionality attached to at least one indicium adapted to capture an image of the body part;
e. wherein a desired axis for the image is defined by the probe; and
f. wherein the imaging functionality may be moved to the correct position and orientation to capture the desired image by alignment with the axis defined by the probe.

20. A system according to claim 19 wherein the imaging functionality is manually positioned.

21. A system according to claim 19 wherein the imaging functionality is automatically positioned.

22. A system according to claim 19 wherein the imaging functionality is a C-arm fluoroscope.

23. A process for conducting computer aided surgery, comprising:
I. providing a computer aided surgery system, comprising:
a. a sensor adapted to sense position of a plurality of indicia attached to an item used in surgery;
b. computer functionality adapted to receive information from the sensor about positions of the indicia and generate information corresponding to position and orientation of the item to which the indicia are attached;
c. a probe adapted to be positioned near a body part, said probe attached to at least one indicium, whereby the position and orientation of the probe is capable of being tracked by said computer functionality;
d. imaging functionality attached to at least one indicium adapted to capture an image of the body part;
e. wherein a desired axis for the image is defined by the probe; and
f. wherein the imaging functionality may be moved to the correct position and orientation to capture the desired image by alignment with the axis defined by the probe;
II. registering the indicia into the system;
III. positioning the probe relative to a desired axis;
IV. storing the position and orientation of the desired axis in the computer functionality;
V. navigating the imaging functionality to the desired axis using the information stored in the computer functionality; and
VI. capturing the desired image.
